(19) United States
(12) Patent Application Publication
(10) Pub. No.: US 2010/0249231 A1
(43) Pub. Date: Sep. 30, 2010

(54) HSP90 Inhibitors of Protein-Protein Interaction HSP90 Chaperone Complexes and Therapeutic Uses Thereof

(75) Inventors: Duxin Sun, Ann Arbor, MI (US); Tao Zhang, Ann Arbor, MI (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/741,430

(22) PCT Filed: Nov. 10, 2008
(86) PCT No.: PCT/US08/82937

§ 371 Date: May 28, 2010

Related U.S. Application Data
(60) Provisional application No. 61/002,610, filed on Nov. 9, 2007.

(51) Int. Cl. A61K 31/19; C12N 5/02; C07C 59/00; C12Q 1/02

(52) U.S. Cl. 514/557; 435/375; 562/461; 435/29

(57) ABSTRACT

A novel Hsp90 inhibitor that disrupts protein-protein interaction in a Hsp90 superchaperone complex without blocking ATP binding and methods for treating diseases such as pancreatic cancer are disclosed.

### CROSS-REFERENCE TO RELATED APPLICATIONS

This application claims the benefit of U.S. Provisional Application Ser. No. 61/002,610 filed Nov. 9, 2007, the entire disclosure of which is expressly incorporated herein by reference.

### STATEMENT REGARDING FEDERALLY SPONSORED RESEARCH

This invention was not made with any Government support and the Government has no rights in this invention.

### TECHNICAL FIELD AND INDUSTRIAL APPLICABILITY OF THE INVENTION

This invention generally relates to novel Hsp90 inhibitors that disrupt protein-protein interaction in a Hsp90 superchaperone complex without blocking ATP binding and methods for treating diseases such as pancreatic cancer.

### BACKGROUND OF THE INVENTION

Pancreatic cancer is the fourth leading cause of cancer death in the United States (1). The treatment option for pancreatic cancer is very limited. The overall 5-year survival rate for pancreatic cancer patients is 4% (2). Currently, targeted therapy has attracted interest for pancreatic cancer therapy. However, the clinical trials using antibodies against EGFR, VEGF, and HER2 in pancreatic cancer had minimal benefits (3-5).

Considering the complexity of pancreatic cancer with multiple genetic abnormalities, targeting a single pathway is unlikely to be entirely effective. Thus, identification of new targets that modulate multiple signaling pathways would be desired for pancreatic cancer therapy.

The molecular chaperone Hsp90 (90-k-Da heat shock protein) may offer many advantages for pancreatic cancer therapy by regulating many oncogenic proteins simultaneously (6, 7). Most of the Hsp90 client proteins are essential for cancer cell survival and proliferation (6). Chaperoning of the Hsp90 client proteins proceeds through a dynamic cycle driven by ATP binding and hydrolysis (8). Hsp90 requires a variety of co-chaperones to form a complex for its function. The co-chaperones bind and release in the super-chaperone complex at various time, including Cdc37, Hsp70, Hsp40, Hop, Hip, p23, pp5, and immunophilins (8).

Hsp90 inhibitors, such as geldanamycin (GA) and its derivative 17-allyamino-geldanamycin (17AAG), bind to the ATP/ADP pocket, arrest Hsp90 in its ADP-bound conformation, thus lead to premature client protein release for the proteasomal degradation to exhibit anticancer activity (8, 9). 17-AAG has entered clinical trial (10, 11).

However, all the current Hsp90 inhibitors bind to the ATP/ADP pocket and completely block the chaperoning function of Hsp90. Up to date, no Hsp90 inhibitor has been approved by FDA to reach market. It is premature to evaluate if the strategy to block the ATP binding of Hsp90 is viable approach for the discovery of Hsp90 inhibitors since many kinases will have similar ATP binding pocket. In addition, many compounds which might inhibit Hsp90 function were probably excluded in the drug screening simply because they cannot fit into ATP binding pocket.

Since the chaperoning process consists of a series of transiently formed complexes (7), halting or inhibiting the chaperoning cycle at various stages is also likely to achieve Hsp90 inhibition.

Thus, the present invention is based, at least in part, on the discovery that a disruption of the Hsp90 and co-chaperone association may be useful to achieve Hsp90 inhibition for pancreatic cancer therapy. Further, this discovery is based, at least in part, in the use of the Cdc37 protein (which is one of the essential co-chaperones of Hsp90 and is a cell division cycle control protein of Saccharomyces cerevisiae). Cdc37 was proposed to be a kinase-specific Hsp90 co-chaperone (12). Although the interaction between Cdc37 and Hsp90 has not yet been fully understood, it is widely accepted that Cdc37 loads client proteins to Hsp90 (13-15).

### SUMMARY OF THE INVENTION

In a first broad aspect, there is provided herein a novel Hsp90 inhibitor that comprises at least one composition that disrupts protein-protein interaction in a Hsp90 superchaperone complex without blocking ATP binding.

In another broad aspect, there is provided herein a novel Hsp90 inhibitor that comprises at least one composition that disrupts Hsp90-Cdc37 interaction in a superchaperone complex without affecting ATP binding of Hsp90.

In certain embodiments, the Hsp90 inhibitor comprises a cyclohexa dienone moiety of the Hsp90 inhibitor is capable of binding to a polar groove of Hsp90 defined by several residues in the lid segment (residues #94 to #125) and the mouth of the nucleotide-binding pocket. In certain embodiments, the hydroxyl and carbonyl groups of the Hsp90 inhibitor are capable of being plugged into a polar and charged pocket surrounded by side chains of Gln119, Glu33, Arg32, and Gly118; and the hydroxyl and carbonyl groups are capable of occupying positions suitable for formation of a H-bond with Glu33 and a NH group of the Gly118 backbone, while the carboxyl moiety of the Hsp90 inhibitor is capable of forming two other H-bonds with side chains of Arg32 and His197.

In certain embodiments, the HSp90 inhibitor comprises a quinone methide triterpene composition. In certain preferred embodiments, the quinone methide triterpene composition comprises celastrol.

In another broad aspect, there is provided herein a method for inhibiting Hsp90 in a subject in need thereof, comprising disrupting protein-protein interaction in a Hsp90 superchaperone complex without blocking ATP binding in the subject.

In another broad aspect, there is provided herein a method for treating a disease condition in a subject, comprising providing a Hps90 inhibitor that disrupts protein-protein interaction in Hsp90 superchaperone complex without blocking ATP binding; and administering the Hsp90 inhibitor to the subject in an amount sufficient to treat the disease condition. In certain embodiments, the disease condition is pancreatic cancer.

In another broad aspect, there is provided herein a method for inhibiting Hsp90 in a subject in need thereof, comprising disrupting Hsp90-Cdc37 complex in at least one cell without affecting the ATP/ADP binding of Hsp90 in the cell.

In another broad aspect, there is provided herein a method for disrupting Hsp90-Cdc37 in a pancreatic cancer cell, comprising degrading Hsp90 client proteins by administering an effective amount of celastrol.

In another broad aspect, there is provided herein a method for treating pancreatic cancer in a subject, comprising administering an effective amount of a Hsp90 inhibitor without affecting ATP/ADT binding of Hsp90 in pancreatic cancer cells.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1D: Molecular docking of celastrol with Hsp90 and Hsp90-Cdc37 complex:

FIG. 1A—Ribbon view and solvent accessible solvent surface of the Hsp90-celastrol binding pocket.

FIG. 1B—Ribbon view of the Hsp90-celastrol binding pocket. Only amino acid residues close to celastrol are displayed for clarity.

FIG. 1C—Ribbon view of the Hsp90-Cdc37-celastrol binding pocket. Only amino acid residues close to celastrol are displayed for clarity.

FIG. 1D—Ribbon view of the superimposition of Hsp90-celastrol (brown) complex with the Hsp90-p23/Sba1 (blue) X-ray structure. The ATP analogue (AMPPNP) is shown and the “lid” segment is colored in yellow for HSP90-celastrol and pink for Hsp90-p23. The p23/Sba1 co-chaperone that is also present in the crystal has been omitted for clarity.

FIGS. 2A-2B: Celastrol disrupts Hsp90-Cdc37 interaction in pancreatic cancer cells:

FIG. 2A—Celastrol does not inhibit ATP binding to Hsp90. γ-Phosphate-linked ATP-Sepharose was used to pull-down Hsp90β in the absence or presence of geldanamycin or celastrol. Hsp90 was detected by Western blot.

FIG. 2B—Celastrol decreases the amount of Cdc37 proteins associated with Hsp90. Panc-1 cells were treated with 10 μM celastrol or GA. Cell lysate was immunoprecipitated with Hsp90 antibody. Western blot was performed for detection of Hsp90, Cdc37, Hop. Cel, celastrol; GA, geldanamycin.

FIGS. 3A-3C: Celastrol does not disrupt Hsp90-p23 complex as GA does:

FIG. 3A—Panc-1 cells treated with 10 μM celastrol or GA. The cell lysate was immunoprecipited with p23 antibody and analyzed for Hsp90.

FIG. 3B—Panc-1 cells were treated with 10 μM celastrol or GA. Cell lysate was immunoprecipitated with Hsp90 antibody and Western blot was performed for detection of p23.

FIG. 3C—Co-chaperone p23 does not coexist with Hop or Cdc37. Panc-1 cell lysates were immunoprecipited by p23 for detection of Hsp90, Hop and Cdc37. Cel, celastrol; GA, geldanamycin.

FIGS. 4A-4C: Effects of celastrol on client proteins degradation and Hsp70 induction:

FIG. 4A—Celastrol and geldanamycin (GA) induces concentration-dependent decreases of Hsp90 client proteins measured by Western blot.

FIG. 4B—Celastrol causes time-dependent decrease of Hsp90 client proteins measured by Western blot.

FIG. 4C—Both Celastrol and GA induce accumulation of Hsp70 protein measured by Western blot.

FIGS. 5A-5B: Celastrol inhibits pancreatic cancer cell proliferation and induces apoptosis:

FIG. 5A—Celastrol exhibits better growth inhibition effect than geldanamycin (GA) by MTS assay.

FIG. 5B—Celastrol (5 μM) induces apoptosis by Annexin-V staining.

FIGS. 6A-6D: Antitumor effects of celastrol in vivo:

FIG. 6A—Antitumor activity of celastrol against Panc-1 xenograft model. vehicle (Vel), Celastrol (Cel) or Geldanamycin (GA). Arrows indicate the dosing time.

FIGS. 6B-6D—Celastrol inhibits tumor growth in RIP1-Tag2 transgenic pancreatic cancer mouse model. Vehicle (Vel), Celastrol (Cel) or Geldanamycin (GA):

FIG. 6B—Comparison of the pancreatic tumor weights and volumes in each group when RIP1-Tag2 mice were sacrificed at the end of drug treatment.

FIG. 6C—Comparison of the metastatic tumor weights and volumes in each group when RIP1-Tag2 mice were sacrificed at week 12.

FIG. 6D—The survival rate of RIP1-Tag2 mice for each drug treatment group.

FIGS. 7A-7B: Molecular docking of celastrol for the interactions with Hsp90-Cdc37 complex:

FIG. 7A—Ribbon view of the Hsp90-Cdc37 X-ray structure with the solvent accessible surface of the Hsp90-Cdc37 interface.

FIG. 7B—Plots of MD-simulated internuclear distances versus the simulation time for Hsp90 binding with celastrol. D1 refers to the H . . . O distance in the hydrogen bond between the hydroxyl group of the ligand and the carboxyl oxygen atom of the Glu-33 residue. D2 refers to the distance between the carbonyl oxygen of the ligand and hydrogen backbone of Gly-118 residue. D3 represents the H . . . N^{δ} distance in the hydrogen bond between the hydroxyl of the carboxylic moiety of the ligand and the His197 residue. D4, represent the internuclear distance between the hydrogen of the guanidinium sidechain of Arg-32 and the carbonyl oxygen of the carboxylic group of the ligand.

FIGS. 8A-8C: Celastrol is different from other proteasome inhibitors:

FIG. 8A—Both celastrol and geldanamycin (GA) induce the accumulation of proteasome client proteins. Panc-1 cells were treated with celastrol and GA. Protein levels of p27 and IκB-α were analyzed by Western blot using specific antibodies.

FIG. 8B—Proteasome inhibitors Mg132 and lactacystin (LCN) do not down-regulate Hsp90 client proteins. Panc-1 cells were treated with increasing concentrations of lactacystin or MG132 for 24 h.

FIG. 8C—Cell viability was analyzed by MTS assay. Panc-1 cells were treated with 10 μM of lactacystin or MG132 for 24 h. Protein levels of Akt and Cdk4 were analyzed by Western blot using specific antibodies.

FIG. 9: Table I. Binding free energies (kcal/mol) calculated at T=298.15 K for human Hsp90 binding with Celastrol ligand.

FIG. 10: RIP1-Tag2 transgenic mouse model of pancreatic islet carcinogenesis [expression of the SV40 large T antigen transgene (Tag) under the rat insulin promoter (RIP)].

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

In a first aspect, it is shown herein that disruption of Hsp90-Cdc37 interaction without affecting the ATP binding of Hsp90 has a similar outcome as Hsp90 inhibition in client protein degradation for pancreatic cancer therapy. Molecular docking, molecular dynamic simulations, and immunoprecipitation confirmed that celastrol[(9b,13a,14b,20a)-3-Hydroxy-9,13-dimethyl-2-oxo-24,25,26-trinoroleana-1(10),3,5,7-tertraen-29-oic acid] (10 μM) disrupted the protein-protein interaction of Hsp90-Cdc37. In contrast to geldanamycin, celastrol (0.1-100 μM) did not interfere with the ATP binding to Hsp90. However, celastrol (1-5 μM) inhibits Hsp90 to induce Hsp90 client protein degradation, similar to classical Hsp90 inhibitor geldanamycin (1-10 μM). It is also shown herein that celastol (IC50=3 μM) exhibited potent anticancer activity against pancreatic cancer cells in vitro. Celastrol (3 mg/kg) inhibited cancer growth by more than 80% in xenograft pancreatic cancer model in vivo. Celastrol (3 mg/kg) also inhibited the tumor metastasis by more than 80% in RIP-tag2 transgenic pancreatic cancer model. It is also shown herein that celastrol disrupts Hsp90-Cdc37 interaction and provides a novel mechanism for Hsp90 inhibition to treat pancreatic cancer.

In a broad aspect, there is provided herein a novel Hsp90 inhibitor that disrupts Hsp90-Cdc37 interaction in the superchaperone complex. The disrupting of Hsp90-Cdc37 interaction without affecting the ATP binding of Hsp90 has a similar outcome as Hsp90 inhibition in client protein degradation for pancreatic cancer therapy.

In another broad aspect, it is shown herein, for the first time, a molecular modeling and experimental confirmation which demonstrates that celastrol, a quinone methide triterpene from tripterygium wilfordii Hook F. (16), inhibited Hsp90 by disrupting Hsp90-Cdc37 complex without affecting the ATP/ADP binding of Hsp90. The disruption of Hsp90-Cdc37 resulted in degradation of Hsp90 client proteins. Celastrol exhibited anticancer activity on pancreatic cancer cells in vitro and in pancreatic xenografts and RIP1-Tag 2 transgenic pancreatic tumor model in vivo.

In another broad aspect, there is provided herein a novel strategy for Hsp90 inhibition for pancreatic cancer therapy.

Targeted therapy appears to be an attractive approach for pancreatic cancer therapy since it specifically targets malfunctioning proteins and signaling pathways (31). However, pancreatic cancer is usually driven by multiple genetic and biochemical abnormalities, targeting only a single pathway might not offer a satisfactory effect. Hsp90 protein may provide advantages by regulating multiple oncogenic proteins (7). In human pancreatic cancer, Hsp90 is expressed 6-7 folds higher compared to normal tissues (32). Recent data reveal that Hsp90 inhibitor (geldanamycin) has displayed anti-tumor effect in nude mice implanted with human pancreatic cancer cells in combination with glycolysis inhibitor (Sun et al, unpublished data).

The Hsp90 protein is a weak ATPase and its function depends on the ability to bind and hydrolyze ATP (19). Two nucleotide-binding domains were identified in Hsp90, located in the N-terminus and C-terminus (8). The early Hsp90 inhibitors target at the N-terminal binding domain, represented by geldanamycin, herbinmycin A and radicicol (9, 33). Subsequently, novobiocin was reported to bind to the C-terminal region of Hsp90 (8, 34). A great deal of effort has been explored to modify geldanamycin to generate a series of derivatives (such as 17-AAG) to reduce its toxicity (35), or designed based on the structure of these naturally occurring compounds, e.g., CCT018159 (36). However, most of the studies were focused to find the Hsp90 inhibitor to block the ATP binding sites (37). Given that no Hsp90 inhibitor has reached market place for human cancer patient, it is premature to conclude if targeting the ATP binding sites is a viable strategy for Hsp90 inhibition.

Now described herein is a novel Hsp90 inhibitor which disrupts protein-protein interaction in the Hsp90 superchaperone complex without blocking ATP binding. Celastrol disrupts Hsp90/Cdc37 complex and leads to the degradation of Hsp90 client proteins. Celastrol exhibits anti-pancreatic tumor activity both in vitro and in vivo.

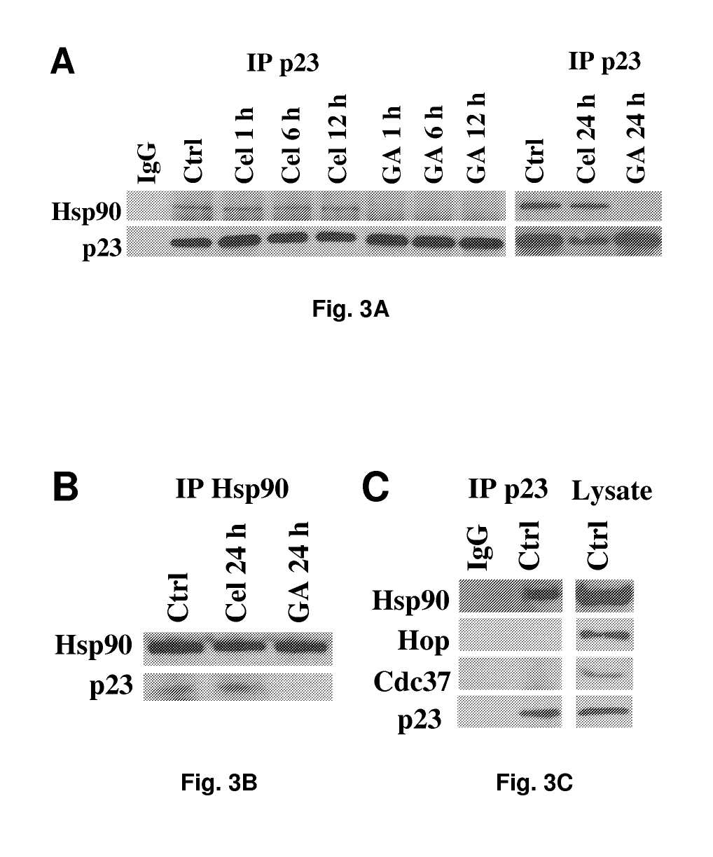

The Hsp90 superchaperone complex has been extensively studied (8, 38). A client protein first binds to Hsp70/Hsp40 complex, then Hop recruits the “open” state Hsp90 to the Hsp70-Hsp40-client complex. The ATP binding to Hsp90 alters its conformation and results in the “closed” state. Hsp70, Hsp40 and Hop are released and replaced by another set of co-chaperones including p23 and immunophilins. With regard to the co-chaperone Cdc37, Hsp70/Hsp40 complex prepares the kinase for interaction with Cdc37, and then Cdc37 recruits Hsp90 to the complex (13, 20, 21). Upon ATP binding, whether Cdc37 dissociates or p23 binds has remained unclear. The inventors' data, using immunoprecipitation with p23 antibody, shows that p23 does not coexist with either Cdc37 or Hop (FIG. 3C).

Structural analysis of Hsp90/Cdc37 reveals that Cdc37 binds to the N-terminal domain of Hsp90 by inserting its C-terminus side chain to the nucleotide binding pocket, holding Hsp90 in an “open” conformation (18). Therefore, progressing from this stage through the ATPase cycle requires the ejection of Cdc37 side chain from the binding pocket (18). In vitro protein binding analysis also suggests that binding of Cdc37 and p23 to Hsp90 is mutually exclusive and the p23-Hsp90-Cdc37 complex could hardly exist (25). Taken together, the data imply that Cdc37 dissociates from Hsp90 superchaperone complex after ATP binding p23 and Cdc37 bind at different stages of the chaperoning cycle.

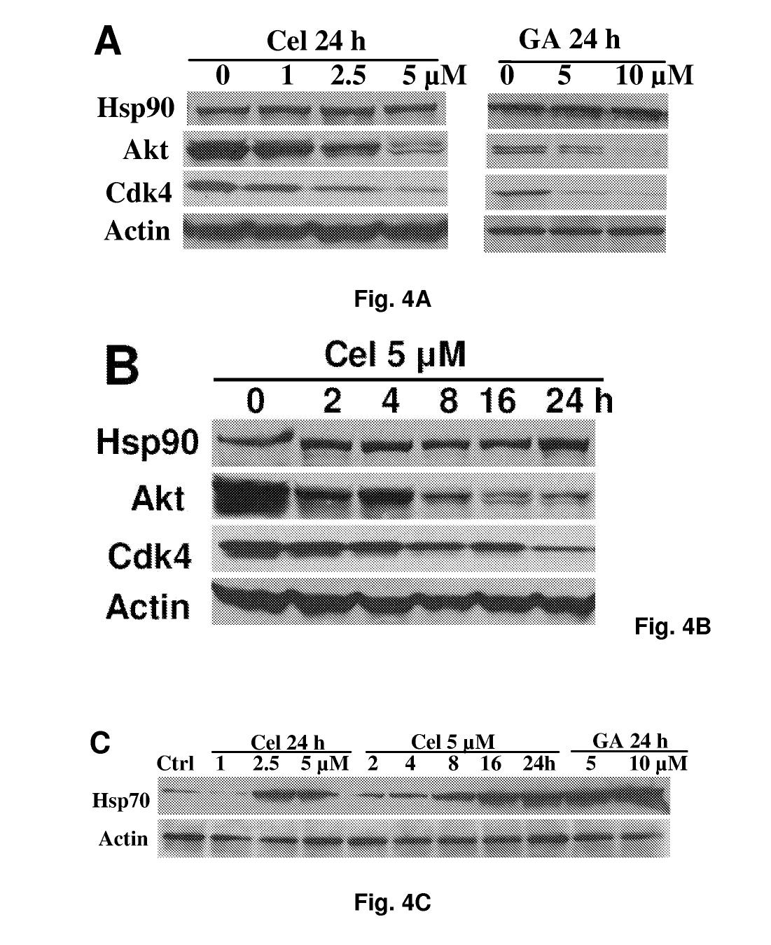

Geldanamycin locks the Hsp90 in the intermediate complex of the loading phase of client proteins (7). Therefore, geldanamycin effectively halted the chaperoning cycle and led to proteasomal degradation of client protein (7, 8). Consistent with these findings, the inventors' data shows that GA inhibited the binding of ATP to Hsp90. Treatment of pancreatic cancer cells with geldanamycin degraded the Hsp90 client proteins, Akt and Cdk4 (FIG. 4A).

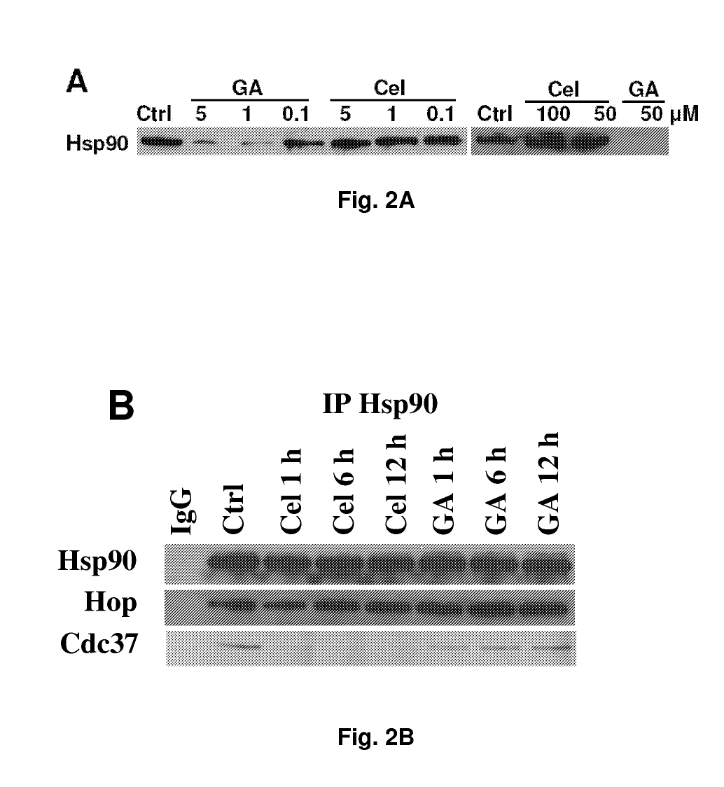

Since GA locks Hsp90 in the intermediate complex, Hsp90 will not have opportunity to bind p23 (22-24). Therefore, GA treatment causes the absence of Hsp90-p23 complex (FIG. 3A and FIG. 3B), which is consistent with previous studies that p23 only binds to ATP-bound form of Hsp90. However, GA did not change either Hsp90-Cdc37 or Hsp90-Hop complex (FIG. 2B). These data show that Hsp90-Cdc37 or Hsp90-Hop coexists in the intermediate complex, which could be arrested by GA.

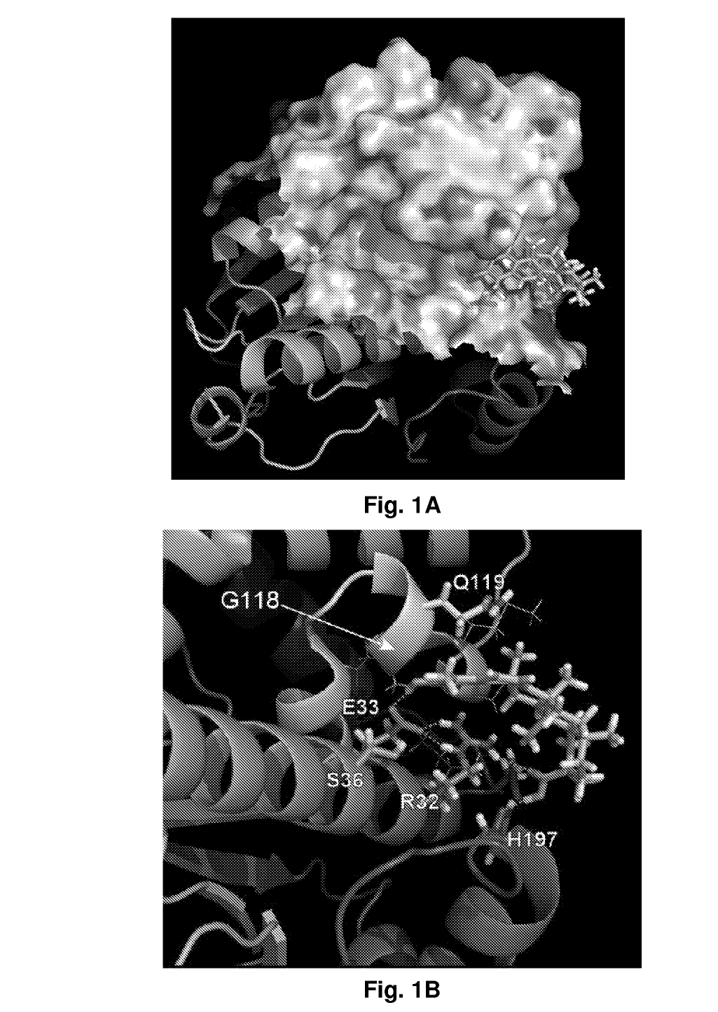

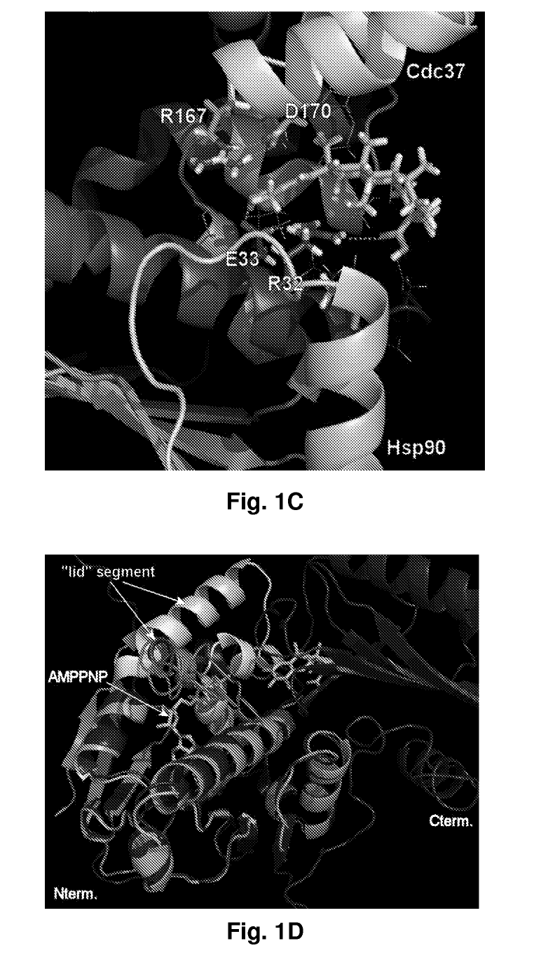

In contrast, celastrol provides a novel mechanism for Hsp90 inhibition. The inventors' computational modeling and co-immunoprecipitation data confirm that celastrol blocks Hsp90-Cdc37 interaction (FIG. 1 and FIG. 2B).

As a consequence, Hsp90 clients are degraded in the time- and dose-dependent manner (FIG. 4A and FIG. 4B). However, celastrol did not interrupt the Hsp90-p23 complex in pancreatic cancer cells (FIG. 3A and FIG. 3B), although different results have been reported in SKBR-3 cells (39). The computer modeling also showed that celastrol could not access the Hsp90/p23 complex since p23 makes celastrol inaccessible to its binding sites in Hsp90. These data explain the sequence of chaperoning cycles in Hsp90 superchaperone complex. The previous results suggested that C-terminus of Cdc37 inserts into the ATP binding pocket of Hsp90 (Open state), ATP binding to Hsp90 ejects Cdc37 from the Hsp90 (18), and sets up the stage for p23 binding to Hsp90. Therefore, Cdc37 and p23 bind to Hsp90 at different stages of chaperoning cycles, and Cdc37 and p23 do not co-exist in one complex. Rather, Cdc37 ejection from Hsp90 seems to be the prerequisite for p23 binding to Hsp90. Therefore, disruption of Hsp90-Cdc37 complex by celastrol may not affect Hsp90-p23 complex. In contrast, it may help to eject Cdc37 from Hsp90 and prepare Hsp90 to bind p23.

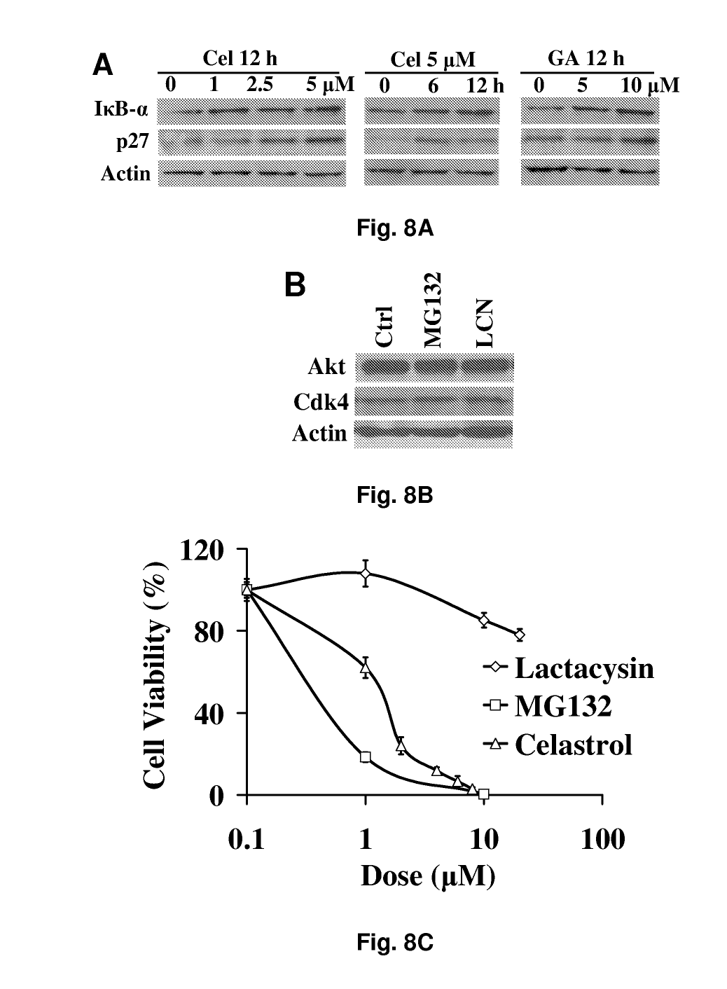

It has been reported that celastrol was a proteasome inhibitor by Yang et al. (28). Consistent with their results, the inventors also confirmed that celastrol induced the accumulation of proteasomal target proteins p27 and IκB-α in Panc-1 cells (FIG. 8A). In addition, the inventors' data also showed that classical Hsp90 inhibitor (geldanamycin) also induced the accumulation of p27 and IκB-α (FIG. 8A). Furthermore, the inventors' data also showed that celastrol is different from other proteasome inhibitors (MG132 and lactacystin). For example, celastrol decreased the levels of Hsp90 client proteins, while proteasome inhibitor MG132 or lactacystin did not change the levels of Hsp90 client proteins (FIG. 8B).

It is now shown herein that celastrol exhibited potent anticancer activity against pancreatic cancer cells in vitro and in xenograft pancreatic cancer in vivo. Celastrol also inhibited the tumor metastasis in RIP-tag2 transgenic pancreatic cancer model. Computer modeling and immunoprecipitation confirmed that celastrol disrupted the protein-protein interaction of Hsp90-Cdc37 for Hsp90 inhibition to induce Hsp90 client protein degradation. In contrast to classical Hsp90 inhibitor geldanamycin, celastrol did not interfere the ATP binding to Hsp90. These data also show that celastrol disrupts Hsp90-Cdc37 interaction and provides a novel mechanism for Hsp90 inhibition to treat pancreatic cancer.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference herein.

### Example I

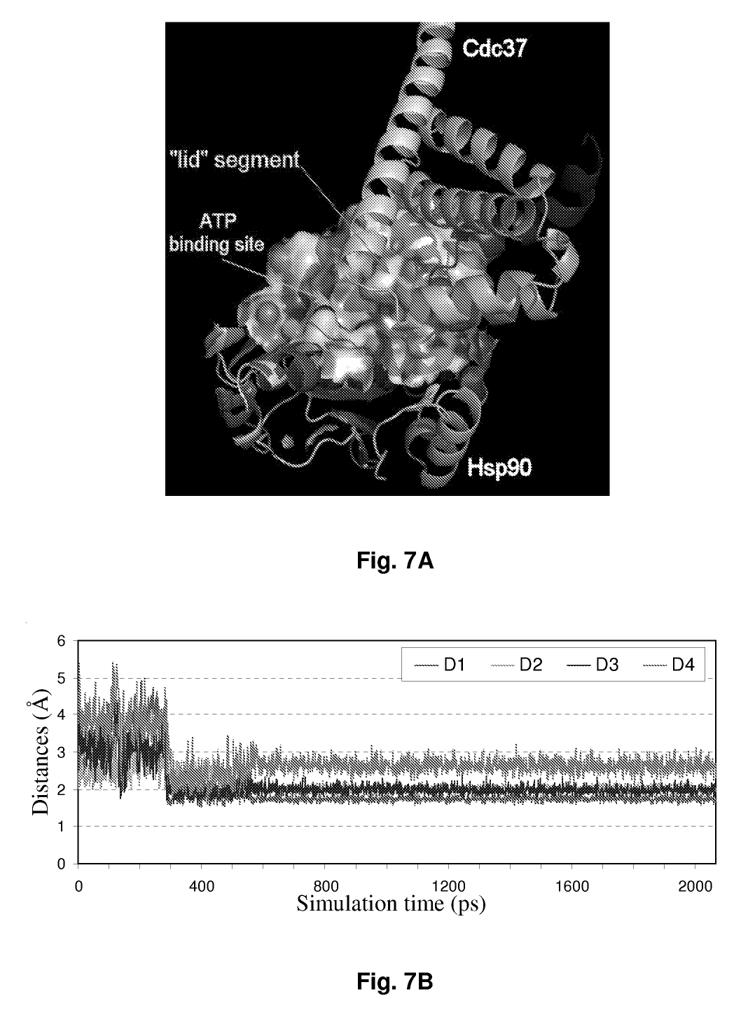

Molecular Docking of Celastrol for the Interactions with the Hsp90 Protein. To investigate whether celastrol disrupts the Hsp90-Cdc37 interaction, the inventors first tested whether celastrol binds Hsp90 residues located either at the interface of the Hsp90-Cdc37 complex or the ATP-binding pocket of Hsp90 (FIG. 7A). An in silico molecular modeling was used to reveal how celastrol binds to Hsp90 and to predict the corresponding Hsp90-ligand binding energy (see Example II below). Both protonated celastrol)(ligand⁰) and deprotonated negatively charged celastrol (Ligand⁻¹) states were used for the molecular docking tests.

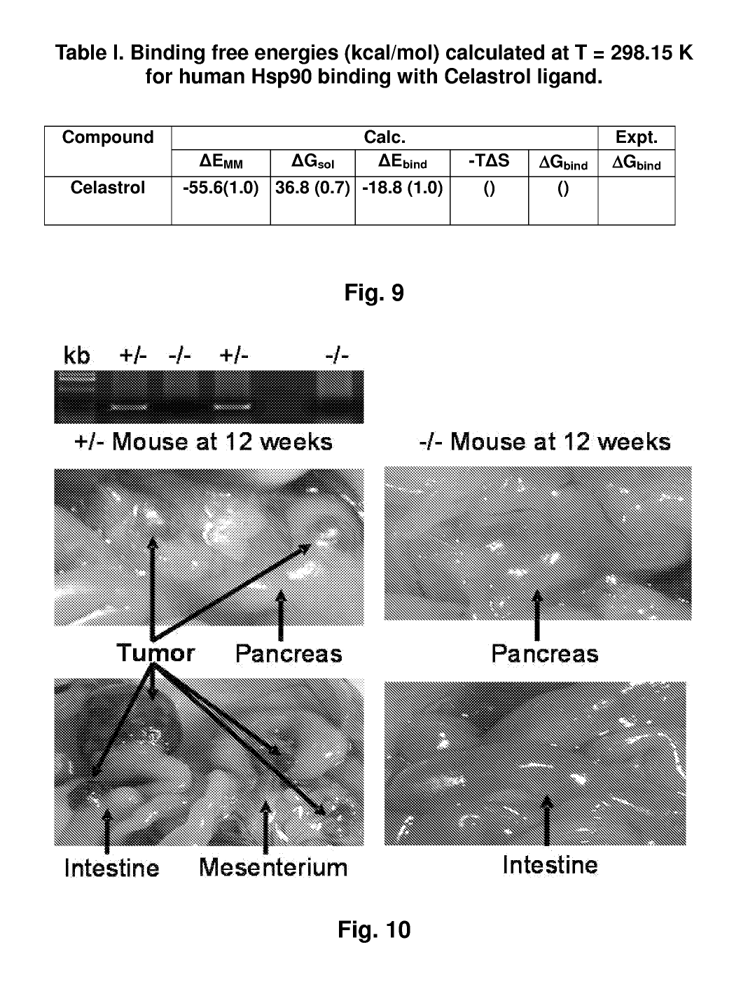

For the Ligand⁻¹ binding with Hsp90, only three possible binding conformations of the Hsp90-Ligand⁻¹ binding complex were found after performing the simulated annealing (SA) procedure. Therefore, the inventors carried out molecular dynamic (MD) simulations on these three possible binding conformations of the Hsp90-ligand⁻¹ binding complex in water (FIG. 9).

The calculated binding energy values range from −1.2 to −4.5 kcal/mol (without accounting for the entropic contributions). In contrast, molecular docking with ligand⁰ produced many possible binding conformations. The 50 most favorable conformations of the ligand⁰ interacting with Hsp90 were used to perform the same simulated annealing procedure. The top 10 conformations were found to interact on the Hsp90 surface of the Hsp90-Cdc37 interface, whereas in the other conformations ligand⁰ binds to the ATP-binding pocket with a significantly lower binding affinity. The binding energy calculation was performed on the top 10 binding conformations using the obtained stable MD trajectories. The most favorable binding conformation of the ligand⁰ binding with Hsp90 has a binding energy of −18.8 kcal/mol, whereas the others have the binding energies between −6 to −11 kcal/mol. All of bind energy calculations ignored the contributions from the entropy, as the inventors were only interested in the relative binding energies and the entropic contributions are not expected to dramatically change the relative binding free energies for a same ligand binding with a same protein (17).

The in silico model of the Hsp90-celastrol binding and the subsequent MD simulation showed that celastrol forms a favorable complex with the Hsp90 in a binding site other than the ATP-binding pocket (FIG. 1A and FIG. 1B).

MD trajectory of the optimum docking model revealed that the distances between celastrol and the key residues of the Hsp90 binding site were found to be stable after the first 0.5 ns of the MD simulation (FIG. 7B).

The averaged structure obtained from the constrained MD simulation is depicted in FIG. 1B. The cyclohexa dienone moiety of celastrol bound to a polar groove of Hsp90 that was defined by several residues in the lid segment (residues #94 to #125) and the mouth of the nucleotide-binding pocket. Thus, the hydroxyl and carbonyl groups of celastrol plugged into a polar and charged pocket surrounded by the side chains of Gln119, Glu33, Arg32, and Gly118. In addition, these groups occupied the positions suitable for the formation of the H-bond with Glu33 and the NH group of the Gly118 backbone, while the carboxyl moiety of celastrol formed two other H-bonds with the side chains of Arg32 and His197.

Additional interactions were also observed between Arg32 and Gln119 residues and the core structure of celastrol. Celastrol was stabilized in the binding site by a π-π stacking interaction (face to face) between the side chain of Arg32 and the aromatic ring of the cyclohexa dienone moiety. A dipole-quadrupole interaction was also observed with the side chain of Glu119 residue.

Molecular Modeling of the Binding between the Hsp90-Celastrol Complex and Cdc37.

It was then determined whether the mode of celastrol binding to the new binding site of Hsp90 can disrupt the Hsp90-Cdc37 binding. The mechanism of Hsp90 ATPase inhibition by the co-chaperone Cdc37 has been well understood at the molecular level (18). Indeed, the interactions between Cdc37 and Hsp90 consist of a substantially hydrophobic interface involving the large helical domain of Cdc37, which packs against the surface of the lid segment of the Hsp90 N-terminal domain. The hydrophobic core is reinforced by a network of polar interactions, including a hydrogen bond contact in which the guanidinium side chain of Arg167 of Cdc37 inserts into the mouth of the nucleotide-binding pocket of Hsp90 N-terminal domain to interact with the side chain of the catalytic residue Glu33. As shown in X-ray structure (1US7.pdb) (18), the N_{φ} atoms of Arg32 side chain mediates two H-bonds, one with the carboxylate of Glu33 and the other with the carboxylate of Asp40.

The Hsp90 protein of the Hsp90-Cdc37 complex in the X-ray crystal structure was superimposed and replaced by the Hsp90-celastrol complex obtained previously, and then submitted to a long MD simulation (˜4 ns). The simulated (Hsp90-celastrol)-Cdc37 complex is depicted in FIG. 1C.

Celastrol was in the mouth of the nucleotide-binding pocket. Three strongest H-bonds stabilizing celastrol in this orientation are still formed by the side chain of Arg32, Glu33, and Gly118 residues. Celastrol filled the binding pocket by forming a π-π stacking interaction with Arg32. The carboxylate of celastrol was involved in a network of H-bonds with Arg32 and His197 side chain. The results showed that celastrol induced the major conformational changes observed in the binding pocket to disrupt the essential H-bond interaction between Arg167 of Cdc37 and Glu33 of Hsp90, which is now believed to significantly decrease the binding affinity of Cdc37 co-chaperone to its cognate Hsp90 and, thus disrupt the Hsp90-Cdc37 binding.

For the Hsp90 superchaperone complex, the current understanding for Hsp90-p23 interaction is that p23 binds to the mature complex of Hsp90 (7). To determine whether celastrol prevents the interaction of Hsp90 and p23/Sba1 co-chaperone, the inventors superimposed the Hsp90-celastrol complex with the X-ray structure of the Hsp90-p23 complex (FIG. 1D).

The results showed that the formation of the Hsp90-celastrol complex requires the lid segment residues being close to residues #26 to #33. The Hsp90 protein existed in two remarkably different conformations in the available X-ray crystal structures. In the X-ray crystal structures reported for both the free Hsp90 protein and Hsp90-Cdc37 complex, the lid segment residues were close to residues #26 to #33. In the X-ray crystal structure of the Hsp90-p23 complex, Hsp90 was in a different conformation in which the lid segment was completely displaced and was close to residues #36 to #50. Moreover, the aforementioned celastrol-binding pocket of Hsp90 is filled by the residues #376 to #385 on the Hsp90 C-terminal in the Hsp90-p23 complex. The conformation of Hsp90 in the Hsp90-p23 complex is clearly not suitable for binding with celastrol. Hence, celastrol is now believed not to disrupt the Hsp90-p23 binding.

Celastrol does not Block ATP Binding to Hsp90.

The molecular docking results showed that celastrol disrupt Hsp90-Cdc37 interaction without blocking the ATP binding pocket of Hsp90. To further confirm these docking results and to exclude the possibility that celastrol binds to the ATP binding pocket of Hsp90, the inventors employed the ATP-Sepharose pull-down assay using purified human Hsp90β protein. As shown in FIG. 2A, ATP-sepharose beads successfully pull-down the Hsp90 in the solution. Since geldanamycin was reported to block the ATP binding pocket of Hsp90, geldanamycin (1 μM) GA was able to block more than 90% of Hsp90β-ATP binding. In contrast, celastrol (0.1 to 100 μM) did not have any effect on the ATP binding. This result further evidenced that celastrol functions differently from the classic Hsp90 inhibitors (geldanamycin).

Celastrol Disrupts Hsp90-Cdc37 Interaction in Pancreatic Cancer Cells.

The Hsp90 superchaperone complex has various co-chaperones at different stages. Previously, it was believed that Cdc37 and Hop were not in the same complex, and Cdc37 bound to the mature complex of Hsp90 superchaperone (7, 19). Current literature suggested that Hsp90, Cdc37, and Hop co-exist in the intermediate complex (20, 21). To confirm the molecular modeling results that celastrol indeed disrupts the Hsp90-Cdc37 interaction, the inventors performed immunoblot analysis of Cdc37 and Hop after immunoprecipitation of Hsp90.

As shown in FIG. 2B, immunoprecipitation of Hsp90 using Hsp90 antibody indeed pull down the Cdc37 and Hop proteins, as detected by Western blot. These results now show that Hsp90-Cdc37 and Hsp90-Hop indeed have the interactions. Celastrol (10 μM) decreased the amount of Cdc37 dramatically in the immunoprecipitated Hsp90 complex early as 1 hr treatment. As a comparison, celastrol did not change the Hsp90-Hop Interaction, as indicated in the unchanged amount of Hop in the immunoprecipitated Hsp90 complex. In contrast, geldanamycin usually binds to ATP binding pocket of Hsp90 and locks the Hsp90 in the intermediate complex with Hop. Therefore, geldanamycin (10 μM) did not change the level of Hop and Cdc37 in immunoprecipitated Hsp90 complex, while it changed the level of hsp90 client protein levels at the same concentration.

These results indicate that celastrol is different from geldanamycin to inhibit Hsp90 by disruption of Hsp90-Cdc37 interaction. The unchanged levels of Hop and Cdc37 in the immunoprecipitated complex by Hsp90 antibody after geldanamycin treatment also now show that Cdc37 and Hop coexist in the intermediate Hsp90 superchaperone complex.

In contrast, co-chaperone p23 is believed to be in the mature complex of Hsp90 superchaperone after ATP binding (22-24). To further confirm the molecular docking results that Hsp90-p23 complex can not accommodate celastrol binding, the inventors performed Western blot analysis for p23 after immunoprecipitation of Hsp90. Anti-Hsp90 antibody was able to pull down p23, suggesting that Hsp90 interacts with p23. Interestingly, after 24 h celastrol treatment, p23 was still present in the immunoprecipitated Hsp90 complex by anti-Hsp90 antibody (FIG. 3B).

To validate the results, the inventors also performed parallel experiments with geldanamycin. Since geldanamycin locks the Hsp90 complex in the intermediate complex with Hop and Cdc37, Hsp90 will not have chance to bind p23 as the mature complex after geldanamycin treatment. Indeed, geldanamycin treatment excluded p23 in the immunoprecipitated Hsp90 complex (FIG. 3B).

To further confirm these results, the inventors conducted Western blot analysis of Hsp90 after immunoprecipitation of p23 by anti-p23 antibody. As shown in FIG. 3A, anti-p23 antibody successfully pulled down the Hsp90, confirming that p23 indeed interacts with Hsp90. However, celastrol did not change the Hsp90 levels in the immunoprecipitated p23 complex by anti-p23 antibody. These results show that celastrol did not disrupt the Hsp90-p23 complex in pancreatic cancer cells. For comparison, geldanamycin (10 μM) treatment for 1 to 24 hrs, immunoprecipitation of p23 by anti-p23 antibody did not pull down any of Hsp90, indicating that p23 is not in the intermediate complex of Hsp90. These data show that celastrol shows different mechanism for Hsp90 inhibition from geldanamycin.

P23 Does not Co-Exist with Cdc37 or Hop.

Since the data showed that celastrol and geldanamycin treatment led to distinct results, which celastrol disrupts Hsp90-Cdc37 in the intermediate complex while geldanamycin was reported to lock Hsp90 in the intermediate complex to exclude Hsp90-p23 interaction, the inventors elucidated the sequence of protein-protein interaction of Hsp90-Cdc37 and Hsp90-p23. Therefore, the immunoprecipitated p23 by anti-p23 antibody in pancreatic cancer cells and then immunoblotted with anti-Cdc37, anti-Hop and anti-Hsp90 antibodies. The results showed that Hsp90 was detected in the immunoprecipitated p23 complex, while neither Hop nor Cdc37 was detected in the precipitated complex with p23 (FIG. 3C).

These results show that both Cdc37 and Hop do not co-exist with p23 in the Hsp90 superchaperone complex. This further confirmed that Cdc37 and Hop are in the intermediate complex of Hsp90 superchaperone while p23 is in the mature complex. Indeed, the data is in agreement with the recent crystal structure of Hsp90/Cdc37 and Hsp90/p23. The structural analysis revealed that Cdc37 binds to the N-terminal nucleotide-binding domain of Hsp90 and arrests the chaperoning cycle in the conformation that could inhibit the binding of p23 to Hsp90 (18). Subsequently, in vitro difference circular dichroism suggests that binding of p23 and Cdc37 to Hsp90 is mutually exclusive (25).

Celastrol Decreases Hsp90 Client Protein Levels.

Since celastrol resulted in the dissociation of Cdc37 from Hsp90, the inventors next determined whether celastrol inhibits Hsp90 function to induce the degradation of its client proteins in pancreatic cells (Panc-1). Panc-1 cells were treated with various concentrations of celastrol or geldanamycin (GA) for different times. The levels of Akt and Cdk4, two well-known Hsp90 clients, were measured. Celastrol decreased the protein levels of Akt and Cdk4 in a concentration- and time-dependent manner. A 24-h treatment of Panc-1 cells with 1 μM celastrol slightly decreased the levels of Akt and Cdk4, but 5 μM celastrol was able to decrease Akt and Cdk4 by 80% and 70%, respectively (FIG. 4A and FIG. 4B). In comparison, GA induced similar levels of Akt and Cdk4 degradation (FIG. 4A).

In addition, Hsp90 inhibitor (geldanamycin) usually does not change the levels of Hsp90, but it will induce Hsp70 protein levels. Indeed, Western blot analysis confirmed that either celastrol or geldanamycin had no effect on Hsp90 protein levels. On the contrary, both celastrol (5 μM) and geldanamycin (5 and 10 μM) was able to induce Hsp70 protein levels by more than 12-fold after 24 h (FIG. 4C).

These data strongly show that celastrol showed similar Hsp90 client protein degradation through disruption of Hsp90-Cdc37 complex when compared to geldanamycin (ATP binding inhibition).

Celastrol Inhibits Cancer Cell Growth and Induces Apoptosis in Pancreatic Cancer Cells.

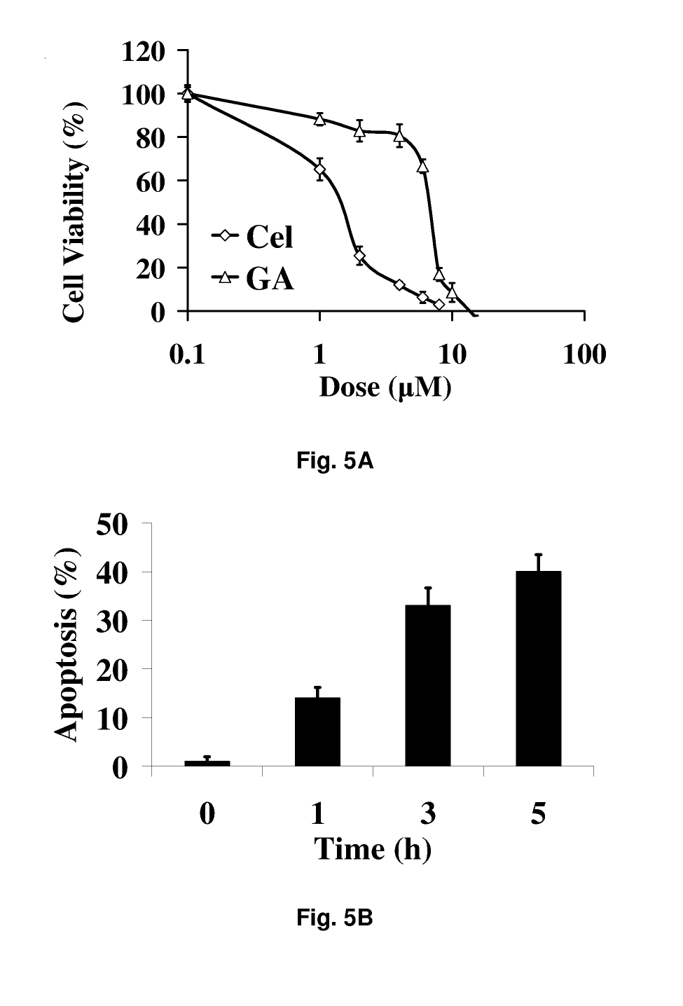

Pancreatic cancer cells are usually resistant to various chemotherapeutic compounds. The inventors selected pancreatic cancer cell line (Panc-1) to test the anticancer effect of celastrol. MTS assay showed that celastrol showed even stronger anticancer activity with IC₅₀ of 3 μM than geldanamycin with IC₅₀ of 8 μM (FIG. 5A).

Annexin-V staining indicated that celastrol (5 μM) induced apoptosis in more than 15% to 40% pancreatic cancer cells after one to five hours incubation (FIG. 5B). Pancreatic cancer cells rounded and congregated when they are incubated with celastrol as early as 1 hour.

Celastrol Shows Strong Anticancer Activity Against Panc-1 Xenograft Tumor.

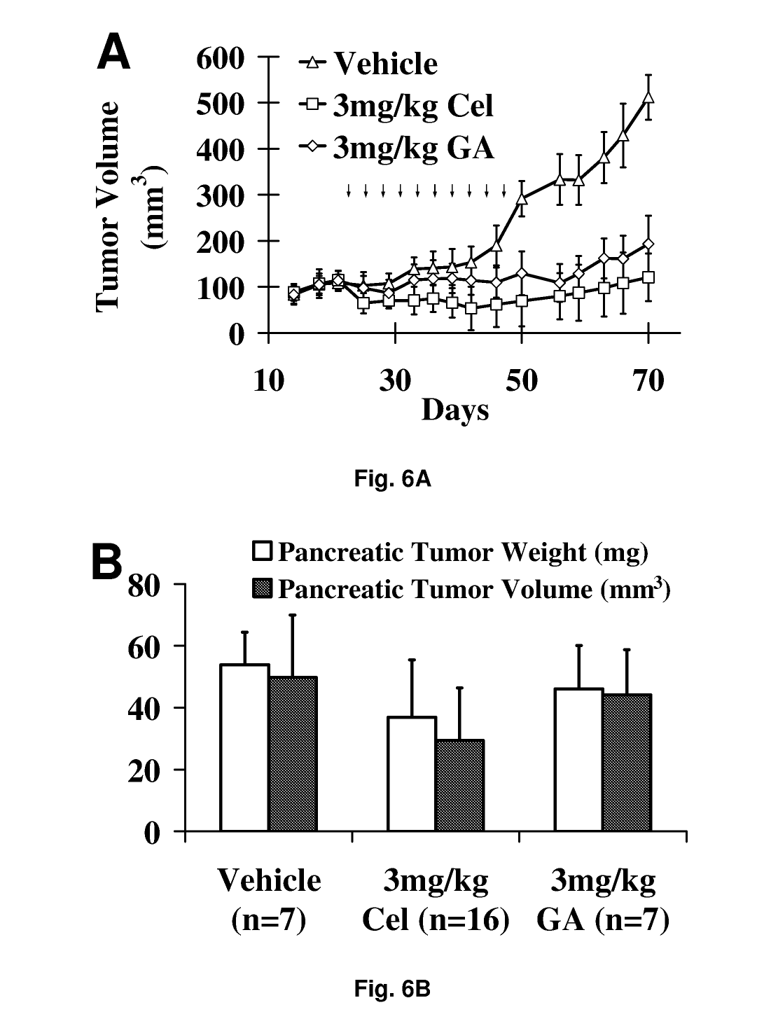

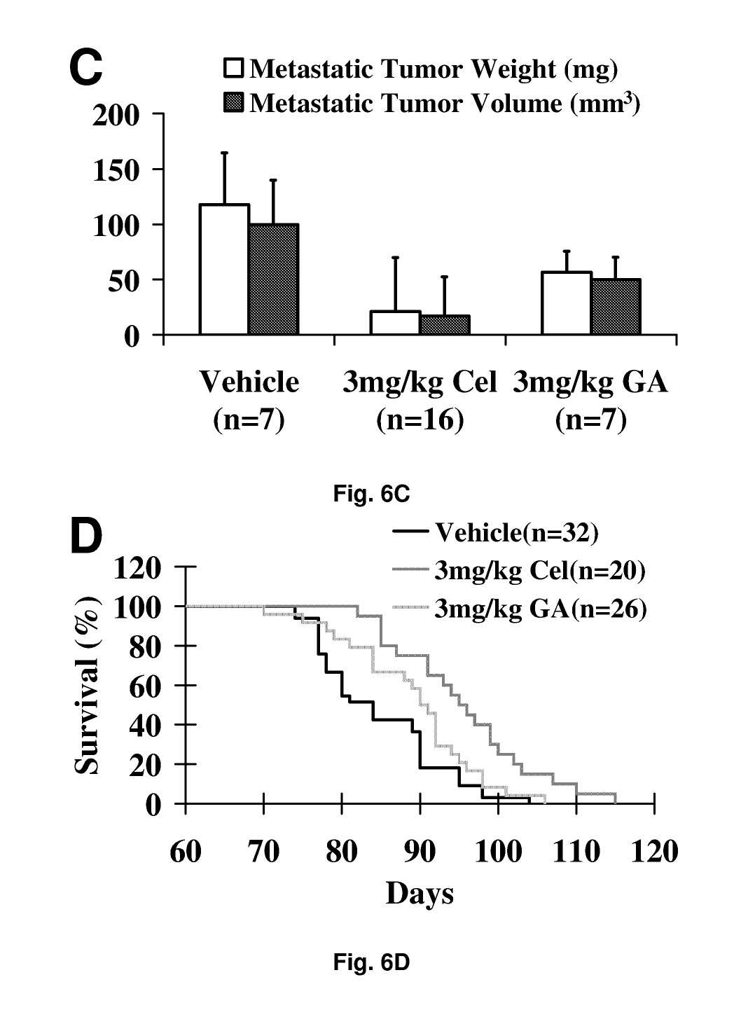

Since celastrol showed anticancer activity against pancreatic cancer cells in vitro, the inventors then tested its anticancer activity in vivo in Panc-1 cell xenograft mice. The mice were injected (i.p.) with vehicle, 3 mg/kg celastrol, 3 mg/kg geldanamycin once per three days for 4 weeks (see Example II below). After the last injection, celastrol inhibited 80% of tumor growth (p<0.001) (FIG. 6A).

The anticancer effect of celastrol was slightly better than that of geldanamycin. Three weeks after the last injection, the average tumor size of the control group reached over 500 mm³, while the tumor size in celastrol treatment group was four to five folds smaller than the control group. Also, it was noted that celastrol treatment exhibited 5% of weight loss in mice (data not show).

Celastrol Inhibits Tumor Growth in RIP1-Tag2 Transgenic Pancreatic Cancer Mouse Model.

To further evaluate the anticancer efficacy of celastrol against pancreatic cancer, the inventors utilized celastrol to inhibit tumor growth in a RIP1-Tag2 transgenic pancreatic cancer mouse model. In this mouse model, hyperplastic islets begin to appear by 3-4 weeks of age, and solid tumors emerge in pancreas at about 8-9 weeks. The cancer will metastasized into other sites such as mesenterium in the peritoneal cavity from 10-12 weeks. Beginning at 8th week, the mice were injected (i.p.) with vehicle, 3 mg/kg celastrol, 3 mg/kg geldanamycin once every 3 days for four weeks (see Example II).

Celastrol decreased metastatic tumors in mesenterium by 80% compared to the control group (P<0.05) and the tumors in pancreas by 30% (P=0.1) at 12th week (FIG. 6B and FIG. 6C). In comparison, geldanamycin inhibited metastatic tumor in mesenterium by 50% (P<0.05) and tumor in pancreas by 13% (P=0.1) at 12th week (FIG. 6B and FIG. 6C).

RIP1-Tag2 transgenic pancreatic cancer mouse only has life span of 11-13 weeks due to pancreatic tumor burden and other symptoms. It is difficult to prolong its survival. Interestingly, the inhibitory effect of celastrol on tumor growth led to a significant prolongation of survival of the RIP1-Tag2 transgenic pancreatic cancer mice. As shown in FIG. 6D, the median survival time for control and celastrol treatment group was 84 and 96 days, representing an average survival increase of 12 days (P<0.001). Geldanamycin treatment group only increased survival by 6 days (P<0.001). These data indicate that celastrol may be useful to inhibit the tumor growth and metastasis of pancreatic cancer. Again, it is noted that celastrol resulted in weight loss for about 10% (data not shown).

FIG. 10 shows RIP1-Tag2 transgenic mouse model of pancreatic islet carcinogenesis [expression of the SV40 large T antigen transgene (Tag) under the rat insulin promoter (RIP)].

Celastrol is Different from Other Proteasome Inhibitors.

To determine whether celastrol also inhibits proteasome for anticancer activity, the inventors measured two well-known target proteins (p27^{Kip1} and IκB-α) of proteasome in pancreatic cancer cells after celastrol treatment (26, 27). A proteasome inhibitor will increase the protein levels of p27 and IκB-α. Interestingly, the results showed that celastrol indeed increased the accumulation of cyclin-dependent kinase inhibitor p27^{Kip1} and IκB-α (FIG. 8A). The data was confirmed by previous studies that celastrol is a proteasome inhibitor and increases levels of proteasomal target proteins (28). In comparison, the classical Hsp90 inhibitor, geldanamycin, also showed similar effect as celastrol for the protein levels of P27 and IκB-α (FIG. 8A). Since celastrol also decreased levels of the Hsp90 client proteins, the inventors compared celastrol and other two well-known proteasome inhibitors (MG132 and lactacystin) for the effect of Hsp90 client proteins (Akt and Cdk4) (29, 30). MTS assay was used to select the effective concentration of MG132 and lactacystin. Interestingly, MG132 or lactacystin (10 μM) did not change the levels of the Cdk4 and Akt proteins (SI FIG. 8B). These results show that celastrol is different from the other proteasome inhibitors.

Material and Methods

Drugs and Reagents. Celastrol was from Cayman Chemical (Ann Arbor, Mich.), proteasome inhibitors MG132 from Calbiochem, Inc. (San Diego, Calif.), and lactacystin from Sigma-Aldrich (St. Louis, Mo.). The following antibodies were used for immunoblotting: Akt, Hsp90 (Cell Signaling, Beverly, Mass.), Hsp70, Hop (StressGen, Victoria, BC, Canada), Cdk4, Cdc37, IκB-α, p27, Hsp90 (Santa Cruz, Santa Cruz, Calif.), actin, p23 (Abcam, Cambridge, Mass.) and monoclonal Hsp90 antibody H9010.

MTS Assay. The human Panc-1 cells were seeded in 96-well plates at a density of 3000-5000 cells per well. Twenty-four hours later the cells were treated with increasing concentrations of either celastrol or geldanamycin as indicated. Number of viable cells was assessed by MTS assay after 24 h. The IC₅₀ values for cytotoxicity were calculated with WinNonlin software (Pharsight, Mountain View, Calif.).

Annexin V-EGFP Assay. Cells treated with 5 μM of celastrol for various time, as well as control cells, were stained with Annexin V-EGFP for analysis of phosphoserine inversion. The Annexin V-EGFP Apoptosis Detection Kit was obtained from BioVision Research Products (Mountain View, Calif.) and was used as recommended by manufacturer.

ATP-Sepharose Binding Assay. The assay was done as previously described (23). 5 μg of human hsp90β protein was pre-incubated on ice for an hour in 200 μl incubation buffer (10 mM Tris-HCl, 50 mM KCl, 5 mM MgCl₂, 2 mM DTT, 20 mM Na₂MoO₄, 0.01% Nonidet P-40, pH 7.5). In competition assays, different concentrations of celastrol and geldanamycin were included in the incubation buffer. Following incubation, 25 μl of pre-equilibrated γ-phosphate-linked ATP-sepharose (JENA BIOSCIENCE) was added and tubes were incubated at 37° C. for 30 min with frequent agitation. Then, the sepharose was pelleted, washed four times with the ice-cold incubation buffer and analyzed by SDS-PAGE.

Western Blot. The general procedure for the Western blot analysis was briefly described as follows. After drug treatment, cells were washed twice with ice-cold phosphate-buffered saline (PBS), scraped into lysis buffer (20 mM Tris, pH 7.5, 1% Nonidet P-40, 150 mM NaCl, 5 mM EDTA, 1 mM Na₃VO₄) supplemented with a protease inhibitor mixture (Sigma, added at a 1:100 dilution), and incubated on ice for 20 min. The lysate was then centrifuged at 14,000×g for 10 min. Equal amounts of total protein were subjected to SDS-PAGE and transferred onto nitrocellulose membranes and probed with appropriate antibodies.

Immunoprecipitation. Cells were washed twice with PBS, lysed in lysis buffer (20 mM Tris HCl (pH 7.4), 25 mM NaCl, 2 mM DDT, 20 mM Na₂MoO₄, 0.1% Nonidet P-40, and protease inhibitors), and incubated at 4° C. for 2 h. After centrifugation at 14,000×g for 10 min, supernatants were incubated with appropriate antibodies at 4° C. for 1 h. Afterwards, protein A/G agarose (Santa Cruz, Santa Cruz, Calif.) was added and further incubated with gentle shaking at 4° C. for 1 h. The agarose was washed five times with lysis buffer, boiled in loading buffer and subjected to SDS-PAGE.

### Example II

Xenograft Mouse Model. 4 to 6-week old nu/nu athymic female mice were obtained from Charles River Laboratories (Charles River, Wilmington, Mass.). The human Panc-1 cells (5-10×10⁶) were mixed with reconstituted basement membrane (Collaborative Research, Bedford, Mass.) and implanted to the right and left flanks of the mice. Tumor volumes were calculated with the formula: width²×length/2. When tumors reached 100 mm³, animals were randomized into different groups for treatment (n=6/group).

Celastrol and geldanamycin were dissolved in the vehicle (10% DMSO, 70% Cremophor/ethanol (3:1), and 20% PBS) (1), and administered at 3 mg/kg by i.p. injection for two continuous days. Then, the dosing schedule was changed to one injection per three days. Tumor sizes and body weights were measured twice a week.

RIP1-Tag2 Transgenic Mouse Model. RIP1-Tag2 transgenic mice used contained the insulin promoter-driven SV40 T-antigen and produced spontaneous multifold and multistage pancreatic islet tumors. RIP1-Tag2 positive mice were identified by PCR analysis of genomic DNA from tail biopsy. RIP1-Tag2 mice of 8 weeks age were randomly divided into 3 groups and injected with vehicle, 3 mg/kg celastrol or geldanamycin once per three days. After 4 weeks of injection, mice of each group were sacrificed by CO₂ and tumors were isolated and saved. The remaining mice were kept to calculate the survival rate.

Molecular docking. The initial coordinates of the human Hsp90 protein used in the computational studies came from the X-ray crystal structure (1US7.pdb) (2) deposited in the Protein Data Bank (3). For comparison, the yeast Hsp90-p23/Sba1 X-ray structure 2CG9.pdb was also used (4).

To explore the possible Hsp90-ligand binding mode, the first step was to identify by virtue the residues in the interface between Hsp90 and Cdc37 in the Hsp90-Cdc37 complex and to dock the ligand to the Hsp90 N-terminal domain. The inventors determined to find where the ligand could be inserted most comfortably. The molecular docking for each possible Hsp90-ligand binding mode was carried out in the same way as the inventors recently performed for studying other protein-ligand binding systems (5). Briefly, a ligand-binding site was defined as that consisting of the residues at the interface of Hsp90-Cdc37 complex and centered on the lid segment of Hsp90 (FIG. 7).

The ligand was initially positioned at ˜10 Å in front of an attempted binding site. The initial docking calculations were performed on the ligand with the N-terminal Hsp90 binding site using the ‘automatic docking’ Affinity module of the Insight package (Acers, Inc.). The Affinity methodology uses a combination of Monte Carlo type and Simulated Annealing (SA) procedures to dock the guest molecule (celastrol) to the host (Hsp90) (6). The Hsp90-ligand binding structure obtained from the initial docking was further refined by performing a molecular dynamics (MD) simulation in water (see below).

Molecular dynamic simulation in water. Each binding complex simulated was neutralized by adding an appropriate number of sodium counter ions and was solvated in a rectangular box of TIP3P water molecules with a minimum solute-wall distance of 10 Å (7). The partial atomic charges for the ligand were obtained after geometry optimizations at the HF/6-31G* level and subsequent single-point calculations of the electrostatic potential, to which the charges were fitted using the RESP procedure (8, 9). Force field parameters of the ligand were assigned based on the atom types of the force field model developed by Cornell et al. (9). Gaussian03 program was used to optimize the geometries and generate electrostatic potentials. The MD simulation was performed by using the Sander module of the Amber8 program (University of California, San Francisco) in a way similar to what we performed for other protein-ligand systems (5). Each of the solvated systems was carefully equilibrated before a sufficiently long MD simulation in room temperature. The MD simulations were performed with a periodic boundary condition in the NPT ensemble at T=298.15 K with Berendsen temperature coupling and constant pressure (P=1 tam) with the isotropic molecule-based scaling. The SHAKE algorithm was applied to fix all covalent bonds containing a hydrogen atom, a time step of 2 fess was used, and the non-bond pair list was updated every 10 steps (10). The particle mesh Weald (PME) method was used to treat long-range electrostatic interactions. A residue-based cutoff of 10 Å was applied to the non-covalent interactions (11).

The obtained stable MD trajectory was used to estimate the binding free energy (ΔG_{rind}) by using the molecular mechanics/Poisson-Boltzmann surface area (MM-PBSA) free energy calculation method (12). 100 snapshots were used to perform the MM-PBSA calculation. MM-PBSA calculation for each snapshot was carried out in the same way as we did for other protein-ligand systems (5). The finally calculated binding free energy was taken as the average of the ΔG_{rind} values with the 100 snapshots.

Most of the MD simulations in water were performed on a HP supercomputer, Superdome, at the Center for Computational Sciences, University of Kentucky. The other computations were carried out on SGI Fuel workstations and a 34-processors IBM x335 Linux cluster in our own lab.

### Example III

### Methods of the Invention

The present invention further provides methods for treating, ameliorating one or more of the symptoms of, and reducing the severity of cancers or neoplastic diseases and related disorders (such as, but not limited to pancreatic cancer) as well as other HSP disorders or conditions.

The methods and compositions of the present invention can be used in the treatment of human cancers. Additionally, compounds of the present invention can be employed as part of a treatment of pancreatic cancer by administering a therapeutically effective amount of at least one of the compounds of the present invention as a single agent or in combination with another anti-cancer agent.

In another broad aspect, there is provided herein a pharmaceutical composition for treating pancreatic cancer, comprising at least one composition that is capable of disrupting protein-protein interaction in a Hsp90 superchaperone complex without blocking ATP binding, or biologically-active fragment thereof, and a pharmaceutically-acceptable carrier.

In another broad aspect, there is provided herein a method of identifying an anti-pancreatic cancer agent, comprising providing a test agent to a cell and measuring the level of a Hsp90 inhibitor that disrupts protein-protein interaction in a Hsp90 superchaperone complex without blocking ATP binding associated with decreased expression levels in pancreatic cancer cells, wherein an increase or a decrease in the level of the Hsp90 inhibitor in the cell, relative to a control cell, is indicative of the test agent being an anti-pancreatic cancer agent.

In another broad aspect, there is provided herein a method of determining the prognosis of a subject with pancreatic cancer, comprising measuring the level of at least one Hsp90 inhibitor in a test sample from the subject, wherein: the Hsp90 inhibitor is associated with an adverse prognosis in pancreatic cancer; and an alteration in the level of the at least one Hsp90 inhibitor in the pancreatic test sample, relative to the level of a corresponding Hsp90 inhibitor in a control sample, is indicative of an adverse prognosis.

In another broad aspect, there is provided herein a method of treating pancreatic cancer in a subject who has a pancreatic cancer in which at least one Hsp90 inhibitor is down-regulated or up-regulated in the cancer cells of the subject relative to control cells, comprising: (1) when the at least Hsp90 inhibitor is down-regulated in the cancer cells, administering to the subject an effective amount of at least one Hsp90 inhibitor, or an isolated variant or biologically-active fragment thereof, such that proliferation of cancer cells in the subject is inhibited; or, (2) when the at least Hsp90 inhibitor is up-regulated in the cancer cells, administering to the subject an effective amount of at least one compound for inhibiting expression of the at least one Hsp90 inhibitor, such that proliferation of cancer cells in the subject is inhibited.

In another broad aspect, there is provided herein a method of treating pancreatic cancer in a subject, comprising: a) determining the amount of at least one Hsp90 inhibitor in pancreatic cancer cells, relative to control cells; and b) altering the amount of Hsp90 inhibitor expressed in the pancreatic cancer cells by: (i) administering to the subject an effective amount of at least one isolated Hsp90 inhibitor, or an isolated variant or biologically-active fragment thereof, if the amount of the Hsp90 inhibitor expressed in the cancer cells is less than the amount of the miR gene product expressed in control cells; or (ii) administering to the subject an effective amount of at least one compound for inhibiting expression of the at least one Hsp90 inhibitor, if the amount of the Hsp90 inhibitor expressed in the cancer cells is greater than the amount of the Hsp90 inhibitor expressed in control cells.

In another broad aspect, there is provided herein a method of identifying an anti-pancreatic cancer agent, comprising providing a test agent to a cell and measuring the level of at least Hsp90 inhibitor associated with an altered expression levels in pancreatic cancer cells, wherein an altered level of the Hsp90 inhibitor in the cell, relative to a control cell, is indicative of the test agent being an anti-pancreatic cancer agent.

In another broad aspect, there is provided herein a method of improving, preventing or treating pancreatic cancer, wherein the method comprises administering a compound comprising celastrol. In certain embodiments, the Hsp90 inhibitor compound can be used in combination with radiation therapy or another anti-cancer chemotherapeutic agent. In certain embodiments, the Hsp90 inhibitor compound can be administered locally to a tumor. In certain embodiments, the Hsp90 inhibitor compound is administered systemically. In certain embodiments, the mode of administration of the Hsp90 inhibitor compound can be inhalation, oral, intravenous, sublingual, ocular, transdermal, rectal, vaginal, topical, intramuscular, intra-arterial, intrathecal, subcutaneous, buccal, or nasal.

In another broad aspect, there is provided herein a method for inhibiting Hsp90 in a cell, comprising contacting a cell expressing an altered amount of Hsp90 with an effective amount of celastrol, or a pharmaceutically acceptable salt thereof. In certain embodiments, the contacting is in vitro. In certain embodiments, the contacting can be in vivo.

In another broad aspect, there is provided herein a kit comprising: a volume a Hps90 inhibitor that disrupts protein-protein interaction in a Hsp90 superchaperone complex in a cell without blocking ATP binding in the cell; and instructions for the use of the volume of Hsp90 inhibitor in the treatment of a disease condition in a mammal. In certain embodiments, the volume of Hsp90 inhibitor is included in a composition that further comprises an additional component selected from the group consisting of a vehicle, an additive, a pharmaceutical adjunct, a therapeutic compound, a carrier, agents useful in the treatment of disease conditions, and combinations thereof.

Treatment of Cancer in Combination with Chemotherapy or Radiotherapy

In certain embodiments, the present invention relates to a method of treating or preventing pancreatic cancer in a subject, comprising administering to a subject in need thereof a therapeutically effective amount of any one or more of the aforementioned compounds.

In certain embodiments, one or more compounds of the present invention are used to treat or prevent cancer or neoplastic disease in combination with one or more anti-cancer, chemotherapeutic agents including, but not limited to, gemcitabine, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, prednisolone, dexamethasone, cytarbine, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. Also, in certain embodiments, one or more compounds of the present invention can be used to treat or prevent cancer or neoplastic disease in combination with one or more chemotherapeutic or other anti-cancer agents including, but not limited to radiation (e.g., gamma-radiation), nitrogen mustards (e.g., cyclophosphamide, Ifosfamide, Trofosfamide, Chlorambucil, Estramustine, and Melphalan), Nitrosoureas (e.g., carmustine (BCNU) and Lomustine (CCNU)), Alkylsulphonates (e.g., busulfan and Treosulfan), Triazenes (e.g., Dacarbazine and Temozolomide), Platinum containing compounds (e.g., Cisplatin, Carboplatin, and oxaliplatin), Vinca alkaloids (e.g., vincristine, Vinblastine, Vindesine, and Vinorelbine), Taxoids (e.g., paclitaxel and Docetaxol), Epipodophyllins (e.g., etoposide, Teniposide, Topotecan, 9-Aminocamptothecin, Camptoirinotecan, Crisnatol, Mytomycin C, and Mytomycin C), Anti-metabolites, DHFR inhibitors (e.g., methotrexate and Trimetrexate), IMP dehydrogenase Inhibitors (e.g., mycophenolic acid, Tiazofurin, Ribavirin, and EICAR), Ribonucleotide reductase Inhibitors (e.g., hydroxyurea and Deferoxamine), Uracil analogs (e.g., Fluorouracil, Floxuridine, Doxifluridine, Ratitrexed, and Capecitabine), Cytosine analogs (e.g., cytarabine (ara C), Cytosine arabinoside, and Fludarabine), Purine analogs (e.g., mercaptopurine and Thioguanine), Anti-estrogens (e.g., Tamoxifen, Raloxifene, and Megestrol), LHRH agonists (e.g., goscrclin and Leuprolide acetate), Anti-androgens (e.g., flutamide and Bicalutamide), Vitamin D3 analogs (e.g., EB 1089, CB 1093, and KH 1060), Photodynamic therapies (e.g., vertoporfin (BPD-MA), Phthalocyanine, photosensitizer Pc4, and Demethoxy-hypocrellin A (2BA-2-DMHA)), Cytokines (e.g., Interferon-alpha, Interferon-gamma., and Tumor necrosis factor), Isoprenylation inhibitors (e.g., Lovastatin), Dopaminergic neurotoxins (e.g., 1-methyl-4-phenylpyridinium ion), Cell cycle inhibitors (e.g., staurosporine), Actinomycins (e.g., Actinomycin D and Dactinomycin), Bleomycins (e.g., bleomycin A2, Bleomycin B2, and Peplomycin), Anthracyclines (e.g., daunorubicin, Doxorubicin (adriamycin), Idarubicin, Epirubicin, Pirarubicin, Zorubicin, and Mitoxantrone), MDR inhibitors (e.g., verapamil), Ca²+ ATPase inhibitors (e.g., thapsigargin), Antibodies (e.g., Avastin, Erbitux, Rituxan, and Bexxar), corticosteroids (e.g., prednilone, predisone, etc), Imatinib, Thalidomide, Lenalidomide, Bortezomib, Gemcitabine, Erlotinib, Gefitinib, Sorafenib, and Sutinib.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents, and observed adverse affects.

Also, in general, compounds of the present invention and the chemotherapeutic agent do not have to be administered in the same pharmaceutical composition, and may, because of different physical and chemical characteristics, have to be administered by different routes. For example, compounds of the present invention may be administered intravenously to generate and maintain good blood levels, while the chemotherapeutic agent may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of chemotherapeutic agent or radiation will depend upon the diagnosis of the physicians and their judgment of the condition of the patient and the appropriate treatment protocol.

A compound of the present invention, and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with a compound of the present invention.

If a compound of the present invention and the chemotherapeutic agent and/or radiation is not administered simultaneously or essentially simultaneously, then the optimum order of administration of the compound of the present invention, and the chemotherapeutic agent and/or radiation, may be different for different tumors. Thus, in certain situations the compound of the present invention may be administered first followed by the administration of the chemotherapeutic agent and/or radiation; and in other situations the chemotherapeutic agent and/or radiation may be administered first followed by the administration of a compound of the present invention. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of a compound of the present invention followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent, i.e., compound of the present invention, chemotherapeutic agent or radiation) of the treatment according to the individual patient's needs, as the treatment proceeds.

Pharmaceutical Compositions

In another embodiment, the present invention provides pharmaceutically acceptable compositions which comprise a therapeutically-effective amount of one or more of the Hsp90 inhibitor compounds described herein, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) pulmonarily, or (9) nasally. As set out above, certain embodiments of the present compounds may contain a basic functional group, such as amino or alkylamino, and are, thus, capable of forming pharmaceutically-acceptable salts with pharmaceutically-acceptable acids.

The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the compounds of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the compounds of the present invention may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the compounds of the present invention in biodegradable polymers such as polylactide-polyglycolide. Depending on the ratio of drug to polymer, and the nature of the particular polymer employed, the rate of drug release can be controlled. Non-limiting examples of other biodegradable polymers include poly(orthoesters) and poly(anhydrides). Depot injectable formulations are also prepared by entrapping the drug in liposomes or microemulsions which are compatible with body tissue.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound may also be administered as a bolus, electuary or paste.

When the compounds are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier. Regardless of the route of administration selected, the compounds, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose will be that amount of the compound which is the lowest dose effective to produce a therapeutic effect. Such an effective dose will generally depend upon the factors described above. Generally, oral, intravenous, intracerebroventricular and subcutaneous doses of the compounds for a patient, when used for the indicated analgesic effects, will range from about 0.0001 to about 100 mg per kilogram of body weight per day. If desired, the effective daily dose of the active compound may be administered as two, three, four, five, six or more sub-doses administered separately at appropriate intervals throughout the day, optionally, in unit dosage forms. Preferred dosing is one administration per day. While it is possible for a compound to be administered alone, it is preferable to administer the compound as a pharmaceutical formulation (composition).

The subject receiving this treatment is any animal in need, including primates, in particular humans, and other mammals such as equines, cattle, swine and sheep; and poultry and pets in general.

The compound can be administered as such or in admixtures with pharmaceutically acceptable carriers and can also be administered in conjunction with antimicrobial agents such as penicillins, cephalosporins, aminoglycosides and glycopeptides. Conjunctive therapy thus includes sequential, simultaneous and separate administration of the active compound in a way that the therapeutical effects of the first administered one is not entirely disappeared when the subsequent is administered.

Disclosed herein are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different carrier molecules are disclosed and discussed, each and every combination and permutation of the carrier molecule and the protease inhibitor are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

In the many inter-related aspects of the inventions disclosed herein, the genera, subgenera, and species of compounds described hereinbelow, including their prodrugs and/or pharmaceutically acceptable salts, and their various pharmaceutical compositions and kits prepared thereof, can be used to treat or prevent pancreatic cancer.

Compounds

The various compounds of the invention disclosed herein comprise a “carrier” molecule and/or the corresponding “carrier” functional group or residues that are either directly or indirectly bonded to another functional group or residue comprising one or more protease inhibitors.

The term “carrier molecule” as defined herein is any compound or functional group or residue thereof that can facilitate the delivery of the protease inhibitor into a muscle tissue. In one aspect, the carrier molecule can be any endogenous molecule. In an alternative embodiment, the carrier molecule can be a derivative of an endogenous compound.

Any of the carrier molecules or residues, linkers, and/or protease inhibitors described herein, and the compounds derived therefrom, can be employed in the form of a pharmaceutical composition, or used to prepare or manufacture pharmaceutical compositions or medicaments.

The pharmaceutical compositions can, where appropriate, be conveniently presented in discrete unit dosage forms and/or kits and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

### DEFINITIONS

As used in the specification and claims, the singular form “a”, “an” and “the” includes plural references unless the context clearly dictates otherwise. For example, the term “a cell” includes a plurality of cells, including mixtures thereof.

The term “compound” as used herein also includes corresponding prodrugs of the compounds of the invention, including actetal prodrugs, and/or one or more pharmaceutically-acceptable salts or esters of the compound and/or prodrugs.

The term “admixing” is defined as mixing the two components, and any additional optional components, together. Depending upon the properties of the components to be admixed, there may or may not be a significant chemical or physical interaction between two or more components when they are mixed. For example, if one component is an acid, and the other component is a base, upon Admixing, the two components may, depending on the strength of the acids and bases, react to form a salt comprising the anion corresponding to the acid and the protonated cation corresponding to the base, or an equilibrium mixture of the original acids and bases, and their salts. In such cases, it will be understood by those of ordinary skill in the art that the resulting composition may be claimed in terms of the components known to be present after the admixing process, or alternatively may be claimed in terms of the components admixed in a product-by-process claim format, especially if the exact nature of the product resulting from the process of admixing the components is unknown or only poorly known or understood.

An “effective amount” of a subject compound, with respect to the present methods of treatment, refers to an amount of the antagonist in a preparation which, when applied as part of a desired dosage regimen brings about, e.g., a change in the rate of cell proliferation and/or rate of survival of a cell according to clinically acceptable standards for the disorder to be treated.

A “patient” or “subject” to be treated by the present method can mean either a human or non-human animal. The term “subject” as used herein, refers to an animal, typically a mammal or a human, that has been the object of treatment, observation, and/or experiment. Also, by “subject” is meant a mammal, including, but not limited to, a human or non-human mammal, such as a bovine, equine, canine, ovine, or feline. In one embodiment, the subject is a human. When the term is used in conjunction with administration of a compound or drug, then the subject has been the object of treatment, observation, and/or administration of the compound or drug.

Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation. By “dysplasia” is meant any abnormal growth or development of tissue, organ, or cells. Preferably, the dysplasia is high grade or precancerous. By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “primary tumor” or “primary cancer” is meant the original cancer and not a metastatic lesion located in another tissue, organ, or location in the subject's body. By “benign tumor” or “benign cancer” is meant a tumor that remains localized at the site of origin and does not have the capacity to infiltrate, invade, or metastasize to a distant site. By “tumor burden” is meant the number of cancer cells, the size of a tumor, or the amount of cancer in the body. Tumor burden is also referred to as tumor load. By “tumor number” is meant the number of tumors.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Non-limiting examples of chemotherapeutic agents include one or more chemical compounds useful in the treatment of cancer. Non-limiting examples of chemotherapeutic agents include alkylating agents such as thiotepa and CYTOXAN®, cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (such as bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK®, polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (such as T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL®, paclitaxel (Bristol-Myers Squibb Oncology, Princeton, N.J.), ABRAXANE®, Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE®, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN®, rIL-2; LURTOTECAN® topoisomerase 1 inhibitor; ABARELIX® rmRH; Vinorelbine and Esperamicins (and pharmaceutically acceptable salts, acids or derivatives of any of the above.

The term “pharmaceutically acceptable salt form” refers to those salt forms that retain the biological effectiveness and properties of the active compound such as sunitinib. Non-limiting examples of such salts include: (1) acid addition salt which is obtained by reaction of the free base of the parent compound with inorganic acids such as hydrochloric acid, hydrobromic acid, nitric acid, 5 phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of sunitinib; or (2) salts formed when an acidic proton present in the parent compound either is replaced by a metal ion, e.g., an alkali metal ion, an alkaline earth ion; or coordinates with an organic base. Exemplary ions include aluminum, calcium, lithium, magnesium, potassium, sodium and zinc in their usual valences. Preferred organic base include protonated tertiary 15 amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine.

The term “prodrug” refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs can include, but are not limited to, phosphate-containing prodrugs, thiophosphate-containing prodrugs, sulfate-containing prodrugs, peptide-containing prodrugs, D-amino acid-modified prodrugs, glycosylated prodrugs, .beta.-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease of 20%, 30%, 40%, 50%, 60%, 70%, 75%, 80%, 85%, 90%, 95%, or greater. Reduce or inhibit can also refer to the symptoms of the disorder being treated, the presence or size of metastases, the size of the primary tumor, or the size or number of the metastatic tumor.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

### REFERENCES

The publication and other material used herein to illuminate the invention or provide additional details respecting the practice of the invention, are incorporated be reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.
1. Jemal, A., Siegel, R., Ward, E., Murray, T., Xu, J. & Thun, M. J. (2007) CA Cancer J Clin 57, 43-66.
2. EVANS, D. B., ABBRUZZESE, J. L. & RICH, T. R. (1997) Cancer Principles and Practice of Oncology, 1054-1087.
3. Kindler, H. L., Friberg, G., Singh, D. A., Locker, G., Nattam, S., Kozloff, M., Taber, D. A., Karrison, T., Dachman, A., Stadler, W. M. & Vokes, E. E. (2005) J Clin Oncol 23, 8033-40.
4. Xiong, H. Q., Rosenberg, A., LoBuglio, A., Schmidt, W., Wolff, R. A., Deutsch, J., Needle, M. & Abbruzzese, J. L. (2004) J Clin Oncol 22, 2610-6.
5. Safran, H., Iannitti, D., Ramanathan, R., Schwartz, J. D., Steinhoff, M., Nauman, C., Hesketh, P., Rathore, R., Wolff, R., Tantravahi, U., Hughes, T. M., Maia, C., Pasquariello, T., Goldstein, L., King, T., Tsai, J. Y. & Kennedy, T. (2004) Cancer Invest 22, 706-12.
6. Neckers, L. (2002) Trends Mol. Med 8, 55-61.
7. Kamal, A., Boehm, M. F. & Burrows, F. J. (2004) Trends Mol Med 10, 283-290.
8. Neckers, L. (2003) Current Medicinal Chemistry 10, 733-739.
9. Whitesell, L., Mimnaugh, E. G., De Costa, B., Myers, C. E. & Neckers, L. M. (1994) Proc Natl Acad Sci USA 91, 8324-8.
10. Supko, J. G., Hickman, R. L., Grever, M. R. & Malspeis, L. (1995) Cancer Chemother. Pharmacol. 36, 305-315.
11. Ramanathan, R. K., Trump, D. L., Eiseman, J. L., Belani, C. P., Agarwala, S. S., Zuhowski, E. G., Lan, J., Potter, D. M., Ivy, S. P., Ramalingam, S., Brufsky, A. M., Wong, M. K. K., Tutchko, S. & Egorin, M. J. (2005) Clin. Cancer Res. 11, 3385-3391.
12. Stepanova, L., Leng, X. H., Parker, S. B. & Harper, J. W. (1996) Genes Dev. 10, 1491-1502.
13. Arlander, S. J., Felts, S. J., Wagner, J. M., Stensgard, B., Toft, D. O. & Karnitz, L. M. (2006) J Biol Chem 281, 2989-98.
14. MacLean, M. & Picard, D. (2003) Cell Stress Chaperones 8, 114-119.
15. Mandal, A. K., Lee, P., Chen, J. A., Nillegoda, N., Heller, A., DiStasio, S., Oen, H., Victor, J., Nair, D. M., Brodsky, J. L. & Caplan, A. J. (2007) J. Cell Biol. 176, 319-328.
16. Setty, A. R. & Sigal, L. H. (2005) Semin Arthritis Rheum 34, 773-784.
17. Hamza, A. & Zhan, C. G. (2006) J Phys Chem B Condens Matter Mater Surf Interfaces Biophys 110, 2910-7.
18. Roe, S. M., Ali, M. M. U., Meyer, P., Vaughan, C. K., Panaretou, B., Piper, P. W., Prodromou, C. and Pearl, L. H. (2004) Cell 116, 87-98.
19. Prodromou, C., Roe, S. M., O'Brien, R., Ladbury, J. E., Piper, P. W. & Pearl, L. H. (1997) Cell 90, 65-75.
20. Lee, P., Shabbir, A., Cardozo, C. & Caplan, A. J. (2004) Mol. Biol. Cell 15, 1785-1792.
21. Pearl, L. H. (2005) Curr Opin Genet Dev 15, 55-61.
22. Johnson, J. L. & Toft, D. O. (1995) Mol Endocrinol 9, 670-678.
23. Grenert, J. P., Sullivan, W. P., Fadden, P., Haystead, T. A., Clark, J., Mimnaugh, E., Krutzsch, H., Ochel, H. J., Schulte, T. W., Sausville, E., Neckers, L. M. & Toft, D. O. (1997) J. Biol. Chem 272, 23843-23850.
24. Sullivan, W., Stensgard, B., Caucutt, G., Bartha, B., McMahon, N., Alnemri, E. S., Litwack, G. & Toft, D. (1997) J Biol Chem 272, 8007-12.
25. Siligardi, G., Hu, B., Panaretou, B., Piper, P. W., Pearl, L. H. & Prodromou, C. (2004) J Biol Chem 279, 51989-51998.
26. Schulte, T. W., An, W. G. & Neckers, L. M. (1997) Biochem Biophys Res Commun 239, 655-659.
27. Verma, I. M., Stevenson, J. K., Schwarz, E. M., Van Antwerp, D. & Miyamoto, S. (1995) Genes Dev. 9, 2723-2735.
28. Yang, H., Chen, D., Cui, Q. C., Yuan, X. & Dou, Q. P. (2006) Cancer Res 66, 4758-65.
29. Kisselev, A. F. & Goldberg, A. L. (2001) Chem Biol 8, 739-58.
30. Fenteany, G., Standaert, R. F., Lane, W. S., Choi, S., Corey, E. J. & Schreiber, S. L. (1995) Science 268, 726-31.
31. Xiong, H. Q. (2004) Cancer Chemother Pharmacol 54 Suppl 1, S69-77.
32. Ogata, M., Naito, Z., Tanaka, S., Moriyama, Y. & Asano, G. (2000) Journal of Nippon Medical School 67, 177-185.
33. Schulte, T. W., Akinaga, S., Soga, S., Sullivan, W., Stensgard, B., Toft, D. & Neckers, L. M. (1998) Cell Stress Chaperones 3, 100-108.
34. Marcu, M. G., Chardli, A., Bouhouche, I., Catelli, M. & Neckers, L. M. (2000) J. Biol. Chem. 275, 37181-37186.
35. Schulte, T. W., Neckers, L. M. (1998) Cancer Chemother. Pharmacol. 42, 273-279.
36. Dymock, B. W., Barril, X., Brough, P. A., Cansfield, J. E., Massey, A., McDonald, E., Hubbard, R. E., Surgenor, A., Roughley, S. D., Webb, P., Workman, P., Wright, L. & Drysdale, M. J. (2005) J. Med. Chem. 48, 4212-4215.
37. Jarvis, L. M. (2007) in Chemical & Engineering News, pp. 15-23.
38. Richter, K. & Buchner, J. (2001) J Cell Physiol 188, 281-290.
39. Hieronymus, H., Lamb, J., Ross, K. N., Peng, X. P., Clement, C., Rodina, A., Nieto, M., Du, J., Stegmaier, K., Raj, S. M., Maloney, K. N., Clardy, J., Hahn, W. C., Chiosis, G. & Golub, T. R. (2006) Cancer Cell 10, 321-30.

### REFERENCES FOR EXAMPLE II

1. Yang, H., Chen, D., Cui, Q. C., Yuan, X. & Dou, Q. P. (2006) Cancer Res 66, 4758-65.
2. Roe, S. M., Ali, M. M. U., Meyer, P., Vaughan, C. K., Panaretou, B., Piper, P. W., Prodromou, C. and Pearl, L. H. (2004) Cell 116, 87-98.
3. Bernstein, F. C., Koetzle, T. F., Williams, G. J., Meyer, E. F., Jr., Brice, M. D., Rodgers, J. R., Kennard, O., Shimanouchi, T. & Tasumi, M. (1977) J Mol Biol 112, 535-42.
4. Ali, M. M., Roe, S. M., Vaughan, C. K., Meyer, P., Panaretou, B., Piper, P. W., Prodromou, C. & Pearl, L. H. (2006) Nature 440, 1013-7.
5. Hamza, A. & Zhan, C. G. (2006) J Phys Chem B Condens Matter Mater Surf Interfaces Biophys 110, 2910-7.
6. Kuntz, I. D., Meng, E. C. & Shoichet, B. K. (1994) Acc. Chem. Res. 27, 117-123.
7. Jorgensen, W. L., Chandrasekhar, J., Madura, J. D., Impey, R. W. & Klein, M. L. (1983) J. Chem. Phys. 79, 926-935.
8. Bayly, C. I., Cieplak, P., Cornell, W. D. & Kollman, P. A. (1993) J. Phys. Chem. 97, 10269-10280.
9. Cornell, W. D., Cieplak, P., Bayl), C. I. & Kollman, P. A. (1993) J. Am. Chem. Soc. 115, 9620-9631.
10. Ryckaert, J. P., Ciccotti, G. & Berendsen, H. J. C. (1977) J. Comput. Phys. 23.
11. Essmann, U., Perera, L., Berkowitz, M. L., Darden, T., Lee, H. & Pedersen, L. G. (1995) J. Chem. Phys. 103, 8577-8593.
12. Kollman, P. A., Massova, I., Reyes, C., Kuhn, B., Huo, S., Chong, L., Lee, M., Lee, T., Duan, Y., Wang, W., Donini, O., Cieplak, P., Srinivasan, J., Case, D. A. & Cheatham, T. E., 3rd (2000) Acc Chem Res 33, 889-97.

1. (canceled)

2. (canceled)

3. (canceled)

4. A method for treating a disease condition in a subject, comprising: providing a Hps90 inhibitor that disrupts protein-protein interaction in Hsp90 superchaperone complex without blocking ATP binding; and administering the Hsp90 inhibitor to the mammal in an amount sufficient to treat the disease condition.

5. The method of claim 4, wherein the disease condition is pancreatic cancer.

6. (canceled)

7. (canceled)

8. (canceled)

9. A method for inhibiting Hsp90 in a subject in need thereof, comprising disrupting Hsp90-Cdc37 complex in a cell without affecting the ATP/ADP binding of Hsp90 in the cell.

10. The method of claim 9, wherein the HSp90 inhibitor comprises a quinone methide triterpene composition.

11. The method of claim 10, wherein the quinone methide triterpene composition comprises celastrol.

12. A method for disrupting Hsp90-Cdc37 in a pancreatic cancer cell, comprising degrading Hsp90 client proteins by administering an effective amount of celastrol.

13. (canceled)

14. A Hsp90 inhibitor, comprising a composition that disrupts protein-protein interaction in a Hsp90 superchaperone complex without blocking ATP binding, wherein a cyclohexa dienone moiety of the Hsp90 inhibitor is capable of binding to a polar groove of Hsp90 defined by several residues in the lid segment (residues #94 to #125) and the mouth of the nucleotide-binding pocket.

15. The Hsp90 inhibitor of claim 14, wherein hydroxyl and carbonyl groups of the Hsp90 inhibitor are capable of being plugged into a polar and charged pocket surrounded by side chains of Gln119, Glu33, Arg32, and Gly118; and
wherein hydroxyl and carbonyl groups are capable of occupying positions suitable for formation of a H-bond with Glu33 and a NH group of the Gly118 backbone,
while the carboxyl moiety of the Hsp90 inhibitor is capable of forming two other H-bonds with side chains of Arg32 and His197.

16. (canceled)

17. A method of identifying an anti-pancreatic cancer agent, comprising
providing a test agent to a cell and measuring the level of a Hsp90 inhibitor that disrupts protein-protein interaction in a Hsp90 superchaperone complex without blocking ATP binding associated with decreased expression levels in pancreatic cancer cells,
wherein an increase or a decrease in the level of the Hsp90 inhibitor in the cell, relative to a control cell, is indicative of the test agent being an anti-pancreatic cancer agent.

18. The method of claim 17, wherein the hsp90 inhibitor comprises celastrol

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)
